(19) United States
(12) Patent Application Publication
(10) Pub. No.: US 2007/0059740 A1
(43) Pub. Date: Mar. 15, 2007

(54) METHOD OF TARGETING A2B ADENOSINE RECEPTOR ANTAGONIST THERAPY

(76) Inventor: Joel M. Linden, Charlottesville, VA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/467,464

(22) Filed: Aug. 25, 2006

Related U.S. Application Data
(60) Provisional application No. 60/711,511, filed on Aug. 26, 2005.

(51) Int. Cl. C12Q 1/68

(52) U.S. Cl. 435/6

(57) ABSTRACT

Provided herein are methods for determining if a subject will benefit from A_{2B} receptor antagonist therapy.

### RELATED APPLICATION

This application claims priority from U.S. Provisional Application Ser. No. 60/711,511 filed Aug. 26, 2005, which application is herein incorporated by reference.

### FIELD OF THE INVENTION

The present invention provides method to select patients, e.g., a diabetic patient or an insulin resistant patient, who can benefit from treatment with an A_{2B} adenosine receptor antagonist.

### BACKGROUND OF THE INVENTION

Insulin is a hormone that regulates the level of blood glucose, and controls the rate at which glucose is transported into fat, liver and muscle cells. In addition, insulin regulates numerous anabolic processes in a variety of other cell types. When excess glucose is transported into fat cells, it is converted to triglycerides that are stored as energy reserves and, eventually, when the stores are needed and insulin is low, the triglycerides are broken down into fatty acids which are either released or converted by the liver into ketones. Insulin actively inhibits breakdown of triglycerides (lipolysis) in fat cells and actively stimulates synthesis of triglycerides from free fatty acids and glucose. Therefore, when insulin levels are low, triglycerides are broken down and the stored fat is lost. Insulin also stimulates glucose uptake into muscle cells, where the glucose is consumed to produce energy or is converted into glycogen, which is a storage form of glucose. In the liver, glucose transport is not insulin sensitive, but conversion of intracellular glucose to glycogen is stimulated by insulin. The liver can convert amino acids to glucose; this process is inhibited by insulin. Binding of insulin by tissue cells depends on insulin receptors on the surface of insulin-sensitive cells. The receptor/insulin complex which extends across the cell membrane transmits signals to the inside of the cell.

These signals increase glucose transport in selected cells and alter cell metabolism in most cells.

Diabetes mellitus is a disease in which the body's metabolism of sugars is greatly impaired due to either impaired secretion of insulin by the pancreas or the body's inability to properly respond to insulin. Diabetes is characterized by elevated levels of glucose in the blood, which can in turn lead to excretion of glucose in urine. Four types of diabetes mellitus have been clinically observed: non-insulin dependent diabetes mellitus (NIDDM); insulin-dependent diabetes mellitus (IDDM); gestational diabetes mellitus (GDM); and diabetes secondary to other conditions. The total incidence of diabetes in the United States population in 1993 was 3.1%, a 500% increase over the incidence of diabetes in 1958. As of 2002, this number has increased to 6.3% of the population.

IDDM, GDM, and secondary diabetes constitute a small portion of the diabetes problem in the United States. Insulin-dependent diabetes is typically manifest as a lack of physiologically functional insulin. IDDM cases typically occur at an early age as a result of autoimmune destruction of the pancreatic β-cells, which are responsible for insulin production. IDDM can also result from cytotoxic destruction of the pancreas, or from errors in insulin synthesis and processing. The most debilitating of diabetic conditions, IDDM fortunately only constitutes approximately 5% of known cases in the United States. Gestational diabetes mellitus is observed in 3%-5% of all pregnancies and typically disappears postpartum. GDM is usually manageable through dietary alterations alone. Diabetes secondary to other conditions (such as sepsis) represents a minor component (1%-2%) of the total cases encountered, but can be serious since it manifests in individuals whose health is already compromised.

The vast majority of diabetics are diagnosed with NIDDM, also commonly referred to as “type II” or “adult-onset” diabetes. In the United States, the incidence of NIDDM is rising sharply. Of the 17 million people characterized as diabetic in the United States in 2002, 90%-95% were considered to be non-insulin dependent diabetics.

The etiology of NIDDM is heterologous. Several genetic syndromes have been associated with the disease. Usually NIDDM is associated with hyperinsulinemia, or excess insulin, rather than a deficiency of insulin. Insulin receptors do not respond to normal levels of insulin, thereby requiring the pancreas to produce greater quantities of insulin. Eventually the pancreas is unable to meet the demand for insulin. Risk factors for NIDDM include older age, family history of diabetes, minority ethnicity, and obesity. Intraabdominal obesity, long duration of obesity, physical inactivity, and morbid obesity, in particular, predispose one to NIDDM.

Chronic hyperglycemia and hyperinsulinemia observed in NIDDM are associated with a large number of health complications. In 2002, 213,062 deaths were attributed to diabetes, not including death from other causes exacerbated by diabetes. Overall, the risk of death among people with diabetes is about 2 times that of people without diabetes.

The complications that arise due to diabetes adversely affect the quality of life of those who suffer from it and result in significant health care costs. General disability affects over 50% of diabetics. Health care services are provided to diabetics with much greater frequency than to age-matched non-diabetics. Vision disorders, especially diabetic retinopathy, afflict over 20% of NIDDM patients. Some form of neuropathy, kidney disease, vascular disease, or cardiovascular disease eventually affects nearly all diabetics. Diabetes patients comprise 35% of all new cases of end stage renal disease. The annual cost of treating diabetes-associated renal disease in the United States exceeds two billion dollars.

Obesity is a risk factor for insulin resistance and diabetes. Obesity is generally defined as a state of being over a normal weight. A person is generally considered to be obese if they are more than about 20% over their ideal weight. That ideal weight must take into account the person's height, age, sex, and body build. Overweight can also be defined as having a body mass index (BMI) of about 25 to about 29.9, obese as having a BMI of about 30 or greater (denoting an excessive accumulation of fat on the body), and morbid obesity is generally defined as a BMI greater than about 40. The incidence and severity of obesity is based upon body mass index (BMI). The prevalence of obesity BMI over 30 and severe obesity (BMI over 40) in the United States is high and rising higher. In the past decade, the overall prevalence rose from 25 to 33%, an increase of 1/3. The deleterious consequences of obesity are considerable. Recent estimates attribute 280,000 deaths a year in the United States to “over-nutrition,” making it second only to cigarette smoking as a cause of death. The prognosis for obesity is poor. Untreated, it tends to progress. With most forms of treatment, weight can be lost, but most persons return to their pretreatment weight within 5 years. The many benefits of even modest weight loss and the difficulty in maintaining weight loss have rekindled interest in the pharmacotherapy of obesity.

Adenosine is a purine breakdown product of ATP that is produced by all cells. It can be transported from the inside of a cell to the outside, where it gains access to adenosine receptors that are located on the outside surface of cells. Adenosine and synthetic compounds that either mimic or block the receptor-mediated actions of adenosine have important clinical applications. Adenosine regulates a wide array of physiological functions, but its effect in any given cell depends on the type of adenosine receptor expressed on the surface of that cell.

The effects of adenosine are mediated by four adenosine receptor subtypes, A₁, A_{2A}, A_{2B}, and A₃. The expression of adenosine receptor subtypes differs from tissue to tissue, and adenosine is thereby able to modulate a variety of physiological effects in a tissue-specific manner. The four known adenosine receptor subtypes interact with GTP-binding proteins (G-proteins) to mediate their effects. Each of the subtypes interacts with a distinct set of G-proteins, and differs in its affinity for different adenosine receptor agonists and antagonists. In addition, a compound can be an agonist or antagonist for more than one of the receptor subtypes; for example, some compounds such as caffeine and theophylline antagonize all four subtypes. The A₁ and A₃ adenosine receptors have been shown to interact primarily with inhibitory G-proteins (Gᵢ), which act to inhibit adenylyl cyclase and reduce intracellular cAMP. The A_{2A} receptor has been shown to elicit an opposite effect, acting through stimulatory G-proteins (Gₛ), to increase adenylyl cyclase activity and increase cAMP. The A_{2B} adenosine receptor is believed to signal similarly through Gₛ (like the A_{2A} receptor), but may also signal through another class of G-proteins (G_{q}) to increase phospholipase C activity, and subsequently, protein kinase C activity. Protein kinase C influences cell metabolism by phosphorylating enzymes and other cell proteins.

Numerous compounds have been reported as functioning as adenosine receptor antagonists. Many uses for these compounds have also been reported. For example, U.S. Pat. Nos. 5,446,046, 5,631,260 and 5,668,139 disclose adenosine and/or xanthine derivatives that function in either the agonism or antagonism of A₁ receptors. Use of these compounds to modulate the biological activity of adenosine through the A₁ receptor, particularly in the treatment of cardiac arrhythmias, is also disclosed.

LaNoue et al. reported the use of a xanthine derivative, particularly 1,3-dipropyl-8-(p-acrylic)-phenylxanthine, as an A_{2B} adenosine receptor antagonist that improves glucose tolerance in Zucker rats (U.S. Pat. No. 6,060,481).

Thus, because of the potentially significant health risks associated with diabetes and insulin resistance, there remains a need for improved methods for identifying, managing and/or treating patients with these and related diseases.

### SUMMARY OF THE INVENTION

The present invention is based upon the discovery that the management and treatment of a type II diabetes patient, an insulin resistant patient, an obesity patient, or a patient having or susceptible to such conditions can be determined based upon an evaluation of the particular patient's ADA genotype, ACP1 genotype, adenosine deaminase (ADA) activity, acid phosphatases locus 1 (ACP1) activity, or any combination thereof.

Thus, the present invention provides a method for identifying a patient, for example, a diabetic patient, an insulin resistant patient and/or an obese patient or a patient at risk of developing diabetes, insulin resistance and/or obesity, who will benefit from A_{2B} adenosine receptor antagonist therapy. In one embodiment, the method involves obtaining a sample from the patient, e.g., a physiological sample with nucleic acid such as a blood sample or tissue sample, and determining the presence of a biomarker in the nucleic acid. Ordinarily, the sample will contain DNA encoding a polymorphic enzyme associated with diabetes, insulin resistance and/or obesity, or the polymorphic enzyme itself. The presence of the molecular biomarker is correlated with the patient benefiting from an A_{2B} adenosine receptor antagonist treatment.

The molecular biomarker comprises nucleic acid encoding an isoform of a polymorphic enzyme that is associated with diabetes, insulin resistance, obesity, high body mass index or a combination thereof, e.g., an enzyme such as adenosine deaminase (ADA) or acid phosphatases locus 1 (ACP1). To illustrate, the biomarker can comprise ADA that is heterozygous for the ADA *1 allele or homozygous for the ADA *2 allele. In another example, the biomarker comprises a polymorphic isoform of the gene encoding ACP1, such as a *B/*B, *A/*C, *B/*C or *C/*C genotype. In yet another embodiment of the invention, the ACP1 genotype is associated with insulin resistance and/or diabetes in the patient. The genotypes of the polymorphic enzymes disclosed herein are also associated with altered enzymatic activity.

One embodiment provides a method for identifying a patient who will benefit from A_{2B} adenosine receptor antagonist therapy, comprising a) obtaining a physiological sample from the patient, wherein the sample comprises a nucleic acid; b) contacting the nucleic acid with at least one oligonucleotide primer; c) subjecting the nucleic acid and at least one oligonucleotide primer to polymerase chain reaction to provide an amplified nucleic acid; and d) determining the presence of a biomarker in the amplified nucleic acid, wherein the biomarker comprises ACP1, wherein the ACP1 genotype is *B/*B, *A/*C, *B/*C or *C/*C and wherein the presence of the genotype is correlated with the patient benefiting from an A_{2B} adenosine receptor antagonist treatment.

In another embodiment of the invention, the method involves measuring the enzymatic activity of a polymorphic enzyme that is present in a physiological sample obtained from the patient, such as a tissue sample, which enzyme is associated with diabetes, insulin resistance, obesity, or a combination thereof. For example, the polymorphic enzyme can be ADA, ACP1 or a combination thereof. Since ADA enzymatically converts adenosine to inosine, individuals expressing an isoform of ADA having low activity are prone to accumulate high levels of adenosine in their tissues. Thus, in one embodiment of the invention a finding of low enzymatic activity of the ADA is indicative of that patient having or likely to develop type II diabetes mellitus, insulin resistance obesity, or a combination thereof. In another example, a finding of high enzymatic activity of ACP1, e.g., it rapidly dephosphorylates and inactivates the insulin receptor, which leads to insulin resistance in the patient, is indicative of that patient having or likely to develop type II diabetes or insulin resistance.

In one embodiment, the A_{2B} adenosine receptor antagonist is a xanthine derivative, an 8-aryladenine or a pharmaceutically acceptable salt thereof. For example, the xanthine derivative includes, but is not limited to, 3-n-propylxanthine, 1,3-dipropyl-8-(p-acrylic)phenylxanthine, 1,3-dipropyl-8-cyclopentylxanthine, 1,3-dipropyl-8-p-sulfophenyl)xanthine, xanthine amine congener, or 1,3-dipropyl-8-[2-(5,6-epoxynorbonyl]xanthine.

Also provided is a method for identifying a patient, e.g., a diabetic, obese or insulin resistant patient, who will benefit from insulin sensitizer therapy, comprising obtaining a physiological sample from the patient, wherein the sample comprises nucleic acid, and determining the presence of a biomarker in the nucleic acid, wherein the biomarker is associated with ACP1 activity. The biomarker can comprise, for example, an ACP1 genotype that is indicative of insulin resistance, such as *B/*B, *A/*C, *B/*C or *C/*C. In one embodiment, the ACP1 activity is medium or high. In another embodiment of the invention, the insulin sensitizer is an A_{2B} adenosine receptor antagonist.

In one embodiment of the method, the treatment selected comprises insulin or drugs that promote insulin secretion.

In another embodiment of the present invention, a method is provided for treating obesity and/or high body mass index, which method employs an A_{2B} adenosine receptor antagonist.

In another embodiment, the present invention provides a kit for identifying the biomarker(s) described herein.

### DETAILED DESCRIPTION OF THE INVENTION

The blockade of A_{2B} adenosine receptors facilitates glucose uptake from blood into skeletal muscle and heart (U.S. Pat. No. 6,060,481). Blocking A_{2B} receptors may also increase glucose uptake into the liver (Yasuda et al., 2003). Overstimulation of A_{2B} receptors on skeletal muscle and/or or liver is associated with low ADA activity, resulting in high adenosine levels. Patients having the polymorphic type of adenosine deaminase (ADA) with low enzymatic activity tend to be obese and at risk for diabetes. A subject with the polymorphic type of acid phosphatases locus 1 (ACP1), an enzyme also known as cytosolic low molecular weight protein tyrosine phosphatases (cLMWPTP) with high enzymatic activity is more likely than a subject with low ACP activity to develop diabetes, and might benefit from A_{2B} blockade.

Thus, the present invention provides a method for guiding the treatment of diabetes, insulin resistance and/or obesity in an individual based upon nucleic acid sequence information obtained from the individual patient. For example, based upon an individual patient's particular ADA and/or ACP1 genotype, i.e., based upon the genotype of the gene encoding ADA and/or ACP1, the patient may benefit from receiving A_{2B} receptor antagonist therapy. A patient's ADA or ACP1 genotype can be determined by methods known to the art, for example, by the gel electrophoresis of red cell lysates (Spencer et al, 1968; Harris and Hopkinson, 1976). The method of the present invention involves obtaining a physiological sample from a patient, which sample contains nucleic acid, identifying the genotype of either the gene encoding ADA, the gene encoding ACP1, or both, and correlating the genotype(s) with a level of ADA enzymatic activity, ACP1 enzymatic activity, or both.

The gene encoding ACP1 has three common codominant alleles, A, B, and C, each of which has a different enzymatic activity (Bottini et al., 1995). The ACP1 enzyme has low activity in A/A and A/B genotypes and high activity in B/B, A/C, B/C, and C/C genotypes. Low ACP1 enzymatic activity is expected to increase insulin signaling in an individual by leaving the insulin receptor in the phosphorylated active state. In fact, subjects with low ACP1 activity have low blood glucose levels (Lucarini et al., 1998; Gloria-Bottini et al., 1996). Thus, a subject with low ACP1 activity, e.g., having the A/A or A/B genotype, is less likely than a subject with high ACP1 activity, e.g., having the B/B, A/C, B/C or C/C genotypes, to manifest insulin resistance and diabetes.

The gene encoding ADA is expressed as two codominant alleles, 1 and 2. High enzymatic ADA activity is associated with the 1/1 genotype, whereas low enzymatic activity is associated with the 1/2 and 2/2 genotypes. Subjects with type 2 diabetes having an ADA genotype with low activity (resulting in high adenosine levels) have a strong tendency to be overweight (i.e. they have a high body mass index) (Bottini and Gloria-Bottini, 1999.) Thus, subjects with low ADA activity (1/2 and 2/2 genotypes) are more likely to respond to A_{2B} adenosine receptor blockers than subjects with high ADA activity (1/1 genotype).

Therefore, using the methods of the present invention it is possible to select the best drug or combination of anti-diabetic drugs based on the subjects ACP1 and ADA genotypes.

I. Definitions

“Acid phosphatase locus 1 (ACP1),” is also known as cytosolic low molecular weight protein tyrosine phosphatase (cLMWPTP or cytosolic low molecular weight PTPase), is a highly polymorphic enzyme that is controlled by a locus on chromosome 2, referred to herein as the ACP1 gene or ACP1. ACP1 is present in two isoforms, ACP1 and ACP1 s.

“ADA” refers to adenosine deaminase, which is a polymorphic enzyme that influences glucose metabolism. In particular, ADA irreversibly deaminates adenosine to inosine, contributing to the regulation of intracellular and extracellular concentrations of adenosine. ADA is constitutively expressed in all tissues investigated. It is deficient in some cases of severe combined immune deficiency (SCID). The gene encoding human ADA has been assigned to chromosome 20 by syntenic analysis using somatic cell hybrids and quantitative enzyme studies on patients with chromosome abnormalities. In situ hybridization of high-resolution somatic and pachytene chromosomes using a 3H-labeled cDNA probe of the ADA gene localized the gene to 20q12—q13.11 (Jhanwar et al., 1989). The gene encoding ADA is referred to herein as ADA or as the ADA gene.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al, (1991); Ohtsuka et al, (1985); Rossolini et al., (1994)).

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins.

The term “nucleotide sequence” refers to a polymer of DNA or RNA that can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

The terms “protein,” “peptide” and “polypeptide” are used interchangeably herein.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

An “allele” is one of several alternative forms of a gene occupying a given locus on a chromosome.

“Polymorphism” refers to the simultaneous occurrence in the population of genomes showing allelic variations (as seen in alleles procuring different phenotypes, for example, enzymes having various levels of activity).

“Biomarker” and “molecular biomarker” refer herein to a marker allele of a gene, e.g., a gene encoding a polymorphic enzyme associated with diabetes, insulin resistance and/or obesity such as ADA or ACP1.

“Genotype” refers to the gene combination at one specific locus or combination of loci. By “genotyping” is meant determining that gene combination using any method known to the art.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. For example, the term variant includes “somatic mutation,” which is a non-heritable DNA change in a part of the body of the affected individual and “germ-line mutation,” which is a DNA alteration originating in sperm or ova that may be passed on to off-spring with the alterations then becoming present throughout the off-spring. Variant nucleotide sequences also include synthetically derived nucleotide sequences, such as those generated, for example, by using site-directed mutagenesis, which encode the native protein, as well as those that encode a polypeptide having amino acid substitutions.

“Genome” refers to the complete genetic material of an organism.

“Expression” refers to the transcription and/or translation of an endogenous gene, heterologous gene or nucleic acid segment, or a transgene in cells. For example, expression refers to the transcription and stable accumulation of sense (mRNA) or functional RNA. Expression may also refer to the production of protein.

By “variant” polypeptide is intended a polypeptide derived from the native protein by deletion (also called “truncation”) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

“Obesity” is defined as (i) a body weight of >30% above ideal or desirable weight on standard height-weight tables; and (ii) in terms of the body mass index (BMI)—weight (in kilograms) divided by the square of the height (in meters).

“Treating” as used herein refers to ameliorating at least one symptom of a disease or a condition.

II. The Association of ACP1 Genotypes with Diabetes, Insulin Resistance and Obesity

As discussed herein, ACP1 is involved in the modulation of signal transduction by insulin, PDGF receptors, and T-cell receptors. High ACP1 activity may increase blood glucose level through depression of insulin action. (Meloni et al, 2003). There is an association between ACP1 genotype and diabetes (see, for example, Meloni et al., 2003; Gloria-Bottini et al., 1996; Lucarini et al., 1998). In addition, a positive association between the low-activity ACP1*A/*A genotype and extreme body mass index was previously shown (Lucarini et al., 1997).

III. The Association of ADA Genotypes with Diabetes, Insulin Resistance and Obesity

A low proportion of the adenosine deaminase (ADA)*2 allele is observed in non-insulin dependent diabetes mellitus (NIDDM) subjects with a body mass index (BMI) of 25 kg/m2 or less, whereas a high proportion of this allele is observed in NIDDM patients with a BMI higher than 34 kg/m2 (Bottini and Gloria-Bottini, 1999). Since the activity of genotypes carrying the ADA*2 allele is lower than that of the more common genotype ADA*1/*1, genetic variability of the enzyme could contribute to degree of obesity in NIDDM (Bottini and Gloria-Bottini, 1999).

Low ADA activity would be expected to result in increased level, of adenosine and increased signaling through adenosine receptors.

IV. Methods of the Present Invention

The enzymatic activity of the polymorphic enzyme(s) can be directly measured from a physiological sample collected from a subject using techniques known to the art. Nucleic acid, such as DNA, can be isolated by blood samples collected from subjects using standard techniques, and DNA encoding the enzyme(s) can be sequenced, for example, using conventional methodology.

Identifying polymorphisms of the invention. Polymorphism of the invention can be identified and analyzed by methods known in the art. For example, allele-specific PCR analysis, PCR-restriction fragment length polymorphism (RFLP) analysis, and allele specific hybridization might be conducted.

For example, in one embodiment of the invention starch gel electrophoresis of red blood lysates is used to genotype the ADA gene (Spencer et al, 1968). Inosine produced by ADA is converted to hypoxanthine by nucleoside phosphorylase and phospate. Hypoxanthine is oxidized by xanthine oxidase, and the tetrazolium salt MTT is reduced in the presence of phenazine methosulphate to a blue insoluble formazan. In ADA *1/*1, there are 3 regularly spaced isozymes with decreased staining intensities in order of their electrophoretic mobilities (anode to cathode). In ADA *2/*2 the pattern is similar, but all three bands electrophorese more slowly. ADA *1/*2 appears as a combination of *1/*1 and *2/*2 with 4 isoforms.

The genotype of the ADA gene can also be determined by DNA sequencing (Yang et al., 1994).

ACP1 genotypes can be determined by starch gel electrophoresis of red blood cell lysates (Miller et al., 1987; Harris and Hopkinson, 1976) or by DNA sequencing techniques (Bryson et al., 1995).

V. A_{2B} Receptor Antagonists

As discussed herein, the methods of the present invention are directed to methods for determining if a subject will benefit from A_{2B} adenosine receptor antagonist therapy. A_{2B} adenosine receptor antagonists are known to the art (including, but not limited to, xanthine or 8-aryladenine derivates), and include, for example, agents disclosed in U.S. Pat. Nos. 6,545,002 and 6,117,878, and U.S. provisional patent application Ser. No. 60/497,875 or Kalla et al., J Med. Chem. 2006; 49(12):3682-92; Godfrey et al., Eur J Pharmacol. 2006; 531(1-3):80-6; Taylor et al., Bioorg Med Chem Lett. 2005; 15(12):3081-5; Gessi et al., Mol Pharmacol. 2005; 67(6):2137-47; Zablocki et al., Bioorg Med Chem Lett. 2005; 15(3):609-12; Stewart et al., Biochem Pharmacol. 2004; 68(2):305-12; Baraldi et al., Bioorg Med Chem Lett. 2004; 14(13):3607-10; Abo-Salem et al., J Pharmacol Exp Ther. 2004; 308(1):358-66; Fozard et al., Eur J Pharmacol. 2003; 475(1-3):79-84; Webb et al., Bioorg Med Chem. 2003; 11(1):77-85; Feoktistov et al., Biochem Pharmacol. 2001; 62(9):1163-73; Ji et al., Biochem Pharmacol. 2001; 61(6):657-63 each of which is incorporated herein by reference for their disclosure of A_{2B} receptor antagonists.

In addition, pharmaceutically acceptable salts of A_{2B} adenosine receptor antagonists may be obtained using standard procedures well known in the art, for example, by reacting a sufficiently basic compound such as an amine with a suitable acid affording a physiologically acceptable anion.

A_{2B} adenosine receptor antagonists can be formulated as pharmaceutical compositions and administered to a mammalian host, such as a human patient in a variety of forms adapted to the chosen route of administration, i.e., orally or parenterally, by intravenous, intramuscular, topical, inhalation or subcutaneous routes.

The present invention also includes kits that comprise one or more reagents, such as oligonucleotide primers, antibodies, enzymes, etc., packaged in sterile condition. Kits of the invention also may include written instructions and other components of the kit.

Thus, the invention provides a kit comprising packing material enclosing, separately packaged, at least one reagent as well as instruction means for their use, in accord with the present methods.

The invention will now be illustrated by the following non-limiting Example.

### EXAMPLE 1

The three codominant alleles of ACP1, i.e., ACP1*A, *B, *C, can be identified by starch gel electrophoresis on red cells hemolyzate or DNA sequencing. The three ACP1 alleles show single base substitutions located at three specific sites: ACPI*A and *B alleles differ by two base substitutions, a silent C-T transition at codon 41 (exon 4) and an A-G transition at codon 105 (exon 6). The ACP1*C allele differs from *A and *B alleles at codon 43 (exon 3).

Total genomic DNA can be extracted from a patient sample, such as a frozen whole-blood sample collected in Na₂EDTA, using procedures known to the art. Polymerase chain reactions can be set up, for example, with 30 microliters, 0.2 μM of each primer, 0.1 mM dNTP's, 1.5 mM MgCl₂, 0.5 Units of Taq polymerase (AmpliTaq, Applied Biosystem), 1×AmpliTaq buffer (PE), and 50 ng of DNA template. The amplification conditions, for example, can consist of an initial denaturation of 94° C. for 2 hours, followed by 35 cycles at 94° C. for 45 minutes, 54° C. for 45 minutes, 72° C. for 45 minutes, and a final extension at 72° C. for 5 hours.

Exemplary oligonucleotide primers that can be used for PCR amplification of the whole blood DNA are in Table 1.

The C-T transition at codon 43 and the A-G transition at codon 105 generate respectively a Cfo I and a Taq I restriction site that, together, can be used for PCR-based genotyping.

TABLE 1

| Primer | Target | Nucleotide sequence |
|---|---|---|
| number | amplification | 5′-3′ |
| #1 | Exon 3 | AGGCCAACCTGAACTCCTCT |
| | | (SEQ ID NO: 1) |
| #2 | Exon 4 | CCTGTCTTGCTTTATGGGCT |
| | | (SEQ ID NO: 2) |
| #3 | Exon 6 | TTCAGAAGACCCTAGCAGATG |
| | | (SEQ ID NO: 3) |
| #4 | Exon 6 | TGGCAAAACCTGCATAACAA |
| | | (SEQ ID NO: 4) |

A 341 bps segment completely spanning exons 3 and 4 can be amplified using primers #1 and #2 (Table 1). A 299 bps segment including exon 5 can be amplified using primers #3 and #4.

Then, 10 microliters of the 341 bps exon 3 amplicon can be cleaved by Cfo I, for example, at 37° C. for 1 hour according to the manufacturer's instructions, and then electrophoresed on 1.8% agarose gels. Such digestion creates two fragments of 255 and 86 bps for ACP1*A and ACP1*B alleles, while the ACP1*C allele is not cut. Similarly, the 299 bps PCR product is digested by Taq I at 65° C. for 1 hour according to the manufacturer's instructions, which generates two fragments of 100 and 199 bps for the ACP1 *A allele, but not for the *B and *C alleles.

### BIBLIOGRAPHY

Bottini and Gloria-Bottini, Metabolism, 48: 949-951 (1999).
Bryson et al., Genomics, 30: 133-140 (1995).
Jhanwar et al., Cytogenet Cell Genet, 50:168-171 (1989).
Gloria-Bottini et al., Experientia, 52, 340-343 (1996).
Lembertas et al., J Clin Invest, 100: 1240-1247 (1997).
Lucarini et al., Hum. Biol., 69, 509-515 (1997).
Lucarini et al., Dis Markers., 14, 121-125 (1998).
Meloni et al., Med Sci Monit, 9: CR105-CR108. (2003)
Miller et al., Hum Hered, 37: 371-375 (1987).
Spencer et al., Ann Hum Genet, 48: pp 49-56 (1968).
Yang et al., Clin. Immunol. Immunopathol., 70:171-175 (1994).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method for identifying a patient who will benefit from A_{2B} adenosine receptor antagonist therapy, comprising
a. obtaining a physiological sample from the patient, wherein the sample comprises a nucleic acid; and
b. determining the presence of a biomarker in the nucleic acid, wherein the presence of the biomarker is correlated with the patient benefiting from an A_{2B} adenosine receptor antagonist treatment.

2. The method of claim 1, wherein the biomarker comprises nucleic acid encoding an isoform of a polymorphic enzyme that is associated with diabetes.

3. The method of claim 1, wherein the biomarker comprises an isoform of adenosine deaminase (ADA).

4. The method of claim 3, wherein the ADA isoform is heterozygous for the ADA *1 allele or homozygous for the ADA *2 allele.

5. The method of claim 1, wherein the biomarker comprises an isoform of ACP1.

6. The method of claim 5, wherein the ACP1 isoform comprises a genotype that is *B/*B, *A/*C, *B/*C, or *C/*C.

7. The method of claim 5, wherein the ACP1 comprises a genotype that is associated with insulin resistance in the patient.

8. The method of claim 5, wherein the ACP1 comprises a genotype that is associated with diabetes in the patient.

9. A method for identifying a patient who will benefit from A_{2B} adenosine receptor antagonist therapy, comprising
a. obtaining a physiological sample from the patient; and
b. measuring the enzymatic activity of a polymorphic enzyme present in the sample, wherein the activity of the polymorphic enzyme is associated with diabetes.

10. The method of claim 9, wherein the polymorphic enzyme comprises adenosine deaminase.

11. The method of claim 10, wherein the adenosine deaminase has low activity.

12. The method of claim 9, wherein the polymorphic enzyme comprises the acid phosphatase locus 1.

13. The method of claim 12, wherein the acid phosphatase locus 1 activity is high.

14. The method of claim 1 or 9, wherein the patient accumulates high levels of endogenous adenosine in their tissue.

15. The method of claim 1 or 9, wherein the patient has or is likely to develop type II diabetes mellitus.

16. The method of claim 1 or 9, wherein the A_{2B} adenosine receptor antagonist is a xanthine derivative, an 8-aryladenine derivative, or a pharmaceutically acceptable salt thereof.

17. The method of claim 16, wherein the xanthine derivative is selected from the group consisting of 3-n-propylxanthine, 1,3-dipropyl-8-(p-acrylic)phenylxanthine, 1,3-dipropyl-8-cyclopentylxanthine, 1,3-dipropyl-8-p-sulfophenyl)xanthine, xanthine amine congener, and 1,3-dipropyl-8-(2-(5,6-epoxynorbonyl) xanthine.

18. A method for identifying a patient who will benefit from insulin sensitizer therapy, comprising
a. obtaining a physiological sample from the patient, wherein the sample comprises nucleic acid; and
b. determining the presence of a biomarker in the nucleic acid, wherein the biomarker is associated with ACP1 activity.

19. The method of claim 18, wherein the biomarker comprises an ACP1 genotype that is associated with insulin resistance.

20. The method of claim 19, wherein the ACP1 genotype is *B/*B, *A/*C, *B/*C, or *C/*C.

21. The method of claim 18, wherein the ACP 1 has high enzymatic activity.

22. The method of claim 18, wherein the insulin sensitizer is an A_{2B} adenosine receptor antagonist.

23. The method of claim 18, wherein the patient is diabetic.

24. The method of claim 21, wherein the treatment selected comprises insulin or drugs that promote insulin secretion.
